(19) United States
(12) Patent Application Publication
(10) Pub. No.: US 2010/0307860 A1
(43) Pub. Date: Dec. 9, 2010

(54) SYSTEMS AND METHODS FOR DONNING A STETHOSCOPE WITH ONE HAND

(76) Inventor: Craig Ellingson, Sandy, UT (US)

Correspondence Address: David B. Tingey, 1800 Eagle Gate Tower, 60 East South Temple, Salt Lake City, UT, 84145, US

(21) Appl. No.: 12/793,626

(22) Filed: Jun. 3, 2010

Related U.S. Application Data
(60) Provisional application No. 61/184,261, filed on Jun. 4, 2009.

(51) Int. Cl. A61B 7/02; B25B 11/00

(52) U.S. Cl. 181/131; 81/485

(57) ABSTRACT

Systems and methods for donning a stethoscope with a single hand. Generally, the systems include a stethoscope having two binaurals and a one-handed, binaural spreading mechanism, which allows a user to force the binaurals apart with a single hand. In some cases, the spreading mechanism includes a first contact member that is attached to a first binaural. In such cases, the spreading mechanism can force the binaurals apart as the first contact member is forced in a binaural spreading direction, which can include forcing the first contact: towards a second contact member that is connected to a second binaural, away from the second contact member, towards a binaural fulcrum that connects the binaurals, away from the fulcrum, and towards the second binaural. Although in some instances, the mechanism retrofits to the stethoscope, in other instances, the mechanism is formed on the stethoscope.

### CROSS-REFERENCE TO RELATED APPLICATION

This application claims priority to U.S. Provisional Patent Application Ser. No. 61/184,261, filed Jun. 4, 2009, and entitled “SYSTEMS AND METHODS FOR DONNING A STETHOSCOPE WITH ONE HAND,” the entire disclosure of which is hereby incorporated by reference.

### BACKGROUND OF THE INVENTION

1. Field of the Invention

The present invention relates to stethoscopes. In particular, the present invention relates to systems and methods for donning a stethoscope on a user through the use of a single hand. By donning the stethoscope with one hand instead of two, the user is able to have a free hand to perform other functions.

2. Background and Related Art

Throughout the world and for many decades, stethoscopes have proven to be extremely useful medical instruments. In many instances, the stethoscope is the first tool that a user, such as a paramedic, doctor, nurse, or other caregiver, utilizes to evaluate the general condition of a new patient.

A stethoscope is a medical device that is used to listen to sounds that are produced within a patient's body. For example, a stethoscope is used to listen to sounds emanating from a patient's heart, lungs, intestines, arteries, veins, and other sound-emanating parts. Stethoscopes typically have a chest piece, air-filled hollow tubing, binaurals/ear tubes, and a pair of ear pieces that are attached at a proximal end of the binaurals. The stethoscopes transmit sound from the chest piece through the hollow tubing, the binaurals, and the ear pieces, directly into the user's ears. In order to help maintain the ear pieces within the user's ears, stethoscopes typically include a spring that extends between the binaurals and acts to bias the binaurals toward each other. Accordingly, once the user has placed the ear pieces within the user's ears (or donned the stethoscope), the spring biases the ear pieces into the user's ears so that the stethoscope remains in the user's ears until the ear pieces are pulled apart and removed from the user's ears.

A stethoscope can be donned when the user grabs each binaural with a separate hand and pulls the binaurals apart so that the space between the two ear pieces increases. The user then places the ear pieces within the user's ears and then releases the binaurals to allow the spring to relax.

While this two-handed donning approach can be used, when the user needs at least one free hand while donning the stethoscope, such an approach is not practical and may be dangerous for a patient being treated by the user. For example, in an emergency situation in which a user, such as a paramedic or an emergency medical technician, is performing an endotracheal intubation to open a patient's airway, the user may need to keep one hand on the tubing that is inserted into the patient's mouth while using the user's other hand to don the stethoscope. In such a situation, if the user were to let go of the tubing to use both hands to don the stethoscope, the tubing can become dislodged from the patient's trachea, move down the patient's esophagus, or otherwise fail to function properly.

Thus, while techniques for donning a stethoscope are available, challenges exist. Accordingly, it would be an improvement in the art to augment or even replace current techniques and apparatuses with other techniques and apparatuses.

### SUMMARY OF THE INVENTION

The present invention relates to stethoscopes. In particular, the present invention relates to systems and methods for donning a stethoscope on a user through the use of a single hand. By donning the stethoscope with one hand instead of two, the user is able to have a free hand to perform other functions.

Implementation of the present invention generally takes place in association with a stethoscope and a one-handed, binaural/ear tube spreading mechanism. With respect to the stethoscope, the stethoscope can have any suitable component. In some instances, the stethoscope comprises two binaurals/ear tubes with an ear piece located on a proximal end of each of the binaurals, a chest piece, a section of flexible tubing extending between the chest piece and a distal end of the binaurals, and a binaural fulcrum that attaches the two binaurals together.

With respect to the one-handed, binaural/ear tube spreading mechanism, the spreading mechanism can comprise virtually any suitable component or characteristic that allows the user to use a single hand to spread the binaurals apart sufficiently so that the user's head can pass between the binaurals' ear pieces. That said, in some instances, the binaural spreading mechanism comprises a first contact member that is configured to be connected to a first binaural as well as to be contacted by a portion of the user's hand, including without limitation, one or more of the user's digits.

The binaural spreading mechanism can function in any suitable manner that allows the user to spread the binaurals apart and to don the stethoscope with a single hand. In some non-limiting implementations, the binaural spreading mechanism is configured to force the binaurals apart when the first contact member is attached to the first binaural and when the first contact member is forced in a binaural spreading direction.

As used herein, the term binaural spreading direction may refer to any suitable direction that causes the binaurals/ear tubes to move away from each other when the first contact member is moved in that direction. In one non-limiting example of a suitable binaural spreading direction, the spreading mechanism causes the binaurals to move away from each other when the first contact member is forced towards a second contact member that is attached to the second binaural. In another non-limiting example, the spreading mechanism is configured to force the binaurals apart when the first contact member is forced away from a second contact member that is attached to the second binaural. In still another non-limiting example, the spreading mechanism is configured to force the binaurals apart as the first contact member is forced towards the binaural fulcrum. In still another non-limiting example, the binaural spreading mechanism is configured to force the binaurals apart as the first contact member is forced away from the binaural fulcrum. In yet another non-limiting example, the spreading mechanism is configured to force the binaurals apart as a portion of the first contact member is forced towards the second binaural.

Where the binaural spreading mechanism is configured to force the binaurals apart as the first contact member is forced towards the second contact member (and vice versa), the spreading mechanism can comprise any suitable component. In some non-limiting examples, the binaural spreading mechanism comprises a first handle and a second handle, a sliding arm that increases in length as the two contact members move towards each other, two pairs of pivoting arms, and/or any other suitable component that comprises the first and second contact members and that is configured to force the binaurals apart when at least a portion of the two contact members are moved towards each other.

Where the binaural spreading mechanism is configured to force the binaurals apart as the first contact member moves away from the second contact member, the spreading mechanism can comprise any suitable component. In some non-limiting examples, the spreading mechanism comprises a pair of contact members that are attached to the binaurals; a sliding arm that increases in length as the contact members move away from each other; and/or any other suitable component that causes the binaurals to move away from each other as a portion of the first and second contact members are forced away from each other.

In implementations in which the binaural spreading mechanism is configured to force the binaurals apart as a portion of the first contact member is foroed towards the binaural fulcrum, the spreading mechanism can comprise any suitable component that allows the spreading mechanism to fulfill its intended purpose. In some non-limiting examples, the spreading mechanism comprises a pivoting contact member that is longer than a distance between the distal-most end of the two binaurals, a contact member that is configured to force the two binaurals apart as the contact member slides distally across the two binaurals, a bendable member that forces the binaurals apart as it is straightened (e.g., a pair of pivoting arms, or a flexible member that is longer than a distance between the distal-most ends of the binaurals), and/or any other suitable component that causes the binaurals to move away from each other as a portion of the first contact member is forced towards the binaural fulcrum.

Where the binaural spreading mechanism is configured to force the binaurals apart as the first contact member is forced away from the binaural fulcrum, the spreading mechanism can comprise any suitable component that allows the spreading mechanism to function as intended. In some non-limiting examples, the binaural spreading mechanism comprises a bendable member that forces the binaurals apart as it is straightened (e.g., a pair of pivoting arms, or a flexible member that is longer than a distance between the distal-most ends of the binaurals), and/or any other suitable component that is capable of forcing the binaurals apart as a portion of that component is forced away from the binaural fulcrum.

In implementations in which the binaural spreading mechanism is configured to force the binaurals apart as a portion of the first contact member moves towards the second binaural, the spreading mechanism can comprise any suitable component. In one non-limiting example, the binaural spreading mechanism comprises a first contact member that connects to the first binaural and which extends laterally past the second binaural. In this example, when a hand-contact portion of the first contact member is forced towards the second binaural, the two binaurals are forced to move in opposite directions. In another non-limiting example, the binaural spreading mechanism comprises a first contact member that includes a first portion and a hand-contact portion, wherein the first portion and the hand-contact portion are mechanically connected through one or more wheels (e.g., gears) that cause the distance between binaurals to increase as the hand-contact portion is forced towards the second binaural.

In addition to the aforementioned embodiments, the one-handed, binaural spreading mechanism can comprise any other suitable component or characteristic. In one non-limiting example the spreading mechanism comprises a first and a second contact member that are formed on the stethoscope in such a manner than when the first and second contact members are forced apart, the first and second binaurals are also forced to move away from each other.

While the systems and methods of the present invention have proven to be particularly useful for donning an acoustic stethoscope on a user in an emergency situation, those skilled in the art can appreciate that the described systems and methods can be used in a variety of different applications and in a variety of different industries. For instance, the skilled artisan can appreciate that the described systems and methods can be used to allow a user to utilize a single hand to don any suitable type of stethoscope (or other device that has ear pieces that are donned in a user's ear), in any other suitable situation.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be understood from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a front view of a stethoscope comprising a representative embodiment of a one-handed, binaural spreading mechanism, wherein the spreading mechanism comprises a first handle and a second handle that are connected to each other by a leaf spring;

FIG. 2 illustrates a front view of a stethoscope comprising a representative embodiment of the one-handed, binaural spreading mechanism, wherein the spreading mechanism comprises the first handle and the second handle, and wherein the handles are connected to each other by a pivot joint;

FIG. 3A illustrates a front view of a stethoscope comprising a representative embodiment of the one-handed, binaural spreading mechanism, wherein the spreading mechanism comprises a sliding arm that is configured to increase in length as a first and a second contact member on the arm are forced towards each other;

FIG. 3B illustrates a top view of the sliding arm from FIG. 3A;

FIG. 4A illustrates a front view of a stethoscope comprising a representative embodiment of the one-handed, binaural spreading mechanism, wherein the spreading mechanism comprises two pairs of pivoting arms;

FIG. 4B illustrates a front view of the spreading mechanism of FIG. 4A, wherein the first contact member and the second contact member have been forced towards each other;

FIG. 5 illustrates a front view of a stethoscope comprising a representative embodiment of the one-handed, binaural spreading mechanism, wherein the first and the second contact members are respectively attached to a first binaural and a second binaural, proximal to an binaural fulcrum;

FIG. 6A illustrates a front view of a stethoscope comprising a representative embodiment of the one-handed, binaural spreading mechanism, wherein the spreading mechanism comprises a sliding arm that is configured to increase in length as the first and the second contact members on the arm are moved away from each other;

FIG. 6B illustrates a top view of the sliding arm from FIG. 6A;

FIG. 7A illustrates a front view of a stethoscope comprising a representative embodiment of the one-handed, binaural spreading mechanism, wherein the first contact member is pivotally attached to the first binaural;

FIG. 7B illustrates a top view of a first contact member from FIG. 7A;

FIG. 8A illustrates a front view of a stethoscope comprising a representative embodiment of the one-handed, binaural spreading mechanism, wherein the spreading mechanism comprises a first contact member that is configured to force the binaurals apart as it slides distally on the binaurals;

FIG. 8B illustrates a top view of the first contact member from FIG. 8A;

FIG. 9 illustrates a front view of a stethoscope comprising a representative embodiment of the one-handed, binaural spreading mechanism, wherein the first contact member comprises a pair of pivoting arms that are configured to separate the binaurals as a portion of the first contact member is forced towards the binaural fulcrum;

FIG. 10A illustrates a front view of a stethoscope comprising a representative embodiment of the one-handed, binaural spreading mechanism, wherein the spreading mechanism comprises the first contact member and the first contact member comprises a flexible portion that is configured to separate the binaurals as a portion of the first contact member is forced towards the binaural fulcrum;

FIG. 10B illustrates a top view of the first contact member from FIG. 10A;

FIG. 11 illustrates a front view of a stethoscope comprising a representative embodiment of the one-handed, binaural spreading mechanism, wherein the first contact member comprises a pair of pivoting arms that are configured to separate the binaurals as a portion of the first contact member is forced away from the binaural fulcrum;

FIG. 12 illustrates a front view of a stethoscope comprising a representative embodiment of the one-handed, binaural spreading mechanism, wherein the spreading mechanism comprises the first contact member, and wherein the first contact member comprises a flexible portion that is configured to separate the binaurals as a portion of the first contact member is forced away from the binaural fulcrum;

FIG. 13 illustrates a front view of a stethoscope comprising a representative embodiment of the one-handed, binaural spreading mechanism, wherein the first contact member connects to the first binaural and extends laterally past the second binaural;

FIG. 14 illustrates a front view of a stethoscope comprising a representative embodiment of the one-handed, binaural spreading mechanism, wherein the spreading mechanism comprises a first portion, which is connected to the first binaural, and a second portion, which is connected to the second binaural; and

FIGS. 15-16 each illustrate a front view of a stethoscope comprising a different representative embodiment of the one-handed, binaural spreading mechanism, wherein the stethoscope has the first and the second contact members formed thereon.

### DETAILED DESCRIPTION OF THE INVENTION

The present invention relates to stethoscopes. In particular, the present invention relates to systems and methods for donning a stethoscope on a user through the use of a single hand. By donning the stethoscope with one hand instead of two, the user is able to have a free hand to perform other functions.

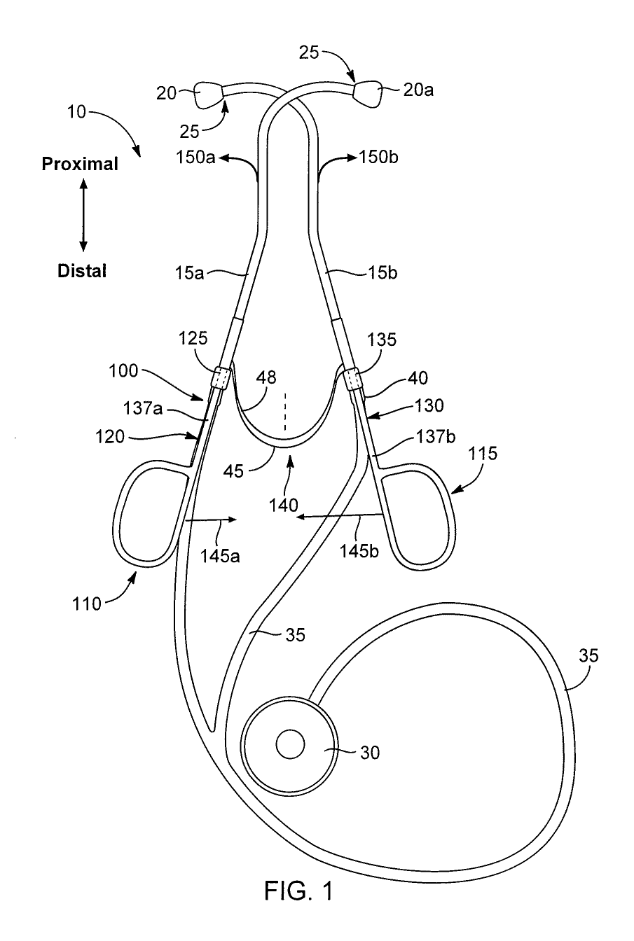

Generally, the described systems and methods can comprise any suitable component that allows a user to don a stethoscope with a single hand. By way of illustration, FIG. 1 shows a non-limiting embodiment in which the described systems and methods comprise a stethoscope 10 and a one-handed, binaural/ear tube spreading mechanism 100.

With respect to the stethoscope 10, the stethoscope can comprise any suitable stethoscope that can be donned on a user with a single hand through the use of the described one-handed, binaural spreading mechanism 100. Some examples of suitable types of stethoscopes include, but are not limited to, acoustic, fetal, recording, noise-reduction, electronic, differential, stereophonic, and any other known or novel type of stethoscope that is suitable for use with the described binaural/ear tube spreading mechanism.

The stethoscope 10 can also comprise any suitable component that allows it to function as intended, as well as to be donned with a single hand through the use of the described binaural spreading mechanism 100. In one non-limiting example, FIG. 1 shows that the stethoscope 10 comprises a pair of binaurals/ear tubes 15, a pair of ear pieces 20 that are disposed at a proximal end 25 of the binaurals 15, a chest piece 30 (such as a diaphragm, a bell, and/or both), tubing 35 (such as a pair of independent acoustic tubes or a single-column, bifurcated tube) that extends between the chest piece 30 and a distal end 40 the binaurals 15, and an binaural fulcrum 45.

With respect to the binaural fulcrum 45, the fulcrum can comprise any suitable component that movably connects a first binaural 15a to a second binaural 15b. In some non-limiting embodiments, the fulcrum comprises a pivot joint, a biasing device (such as one or more internal and/or external leaf springs that bias the binaurals towards each other), tubing that connects to the binaurals, a binaural coupler, and/or any other suitable component that movably joins the binaurals in a manner that allows the user to don the stethoscope. In one non-limiting example, FIG. 1 shows the fulcrum 45 comprises an external leaf spring 48.

Turning now to the one-handed, binaural spreading mechanism 100, the spreading mechanism can comprise any suitable component or characteristic that allows the user to spread the binaurals 15 far enough apart, using a single hand, to pass the user's head between the ear pieces 20. In some non-limiting embodiments, the spreading mechanism comprises one or more contact members. In some non-limiting examples, the spreading mechanism comprises a first contact member and/or a second contact member. As used herein, the term contact member may refer to a component that is adapted to be connected to the first binaural 15a and/or to the second binaural 15b in a manner that causes the binaurals to move in opposite directions when the user applies pressure to the contact member in an binaural spreading direction (or a direction that causes the binaurals to move away from each other when the first contact member is moved in that direction).

Where the binaural spreading mechanism comprises one more contact members, the contact members can have any suitable characteristic that allows them to force the binaurals apart when pressure is applied to one or all of the contact members. In some non-limiting embodiments, each contact member is configured to contact any suitable portion of the user's hand, including, without limitation, one or more of the user's digits (e.g., thumb or fingers), the palm of the user's hand, etc. By way of non-limiting example, the first contact member and the second contact member can each comprise a loop, a finger hold, a post, a protuberance, a catch, a cradled portion, a flat surface, and/or any other suitable contact surface. For instance, FIG. 1 shows a non-limiting embodiment in which the first 110 and second 115 contact members each comprises a loop. FIG. 1 also shows that, in at least some non-limiting embodiments, where the binaural spreading mechanism 100 comprises two contact members (e.g., 110 and 115), one contact member (e.g., the first contact member 110) is configured to contact the user's thumb (not shown) while the other contact member (e.g., the second contact member 115) is configured to contact one or more of the user's other fingers (not shown) on the same hand.

As another non-limiting example of a suitable characteristic of the spreading mechanism 100, in some embodiments in which the mechanism comprises the first 110 and the second 115 contact members, each contact member is configured to be right-handed, left-handed, or ambidextrous. By way of illustration, FIG. 1 shows a non-limiting embodiment in which the first 110 and second 115 contact members each comprises an ambidextrous loop.

Moving now to the functioning of the binaural spreading mechanism 100, the spreading mechanism can function in any suitable manner. Where the spreading mechanism comprises the first contact member, the mechanism can force the binaurals 15 apart when the first contact member is forced in any suitable binaural spreading direction. In some non-limiting embodiments, the spreading mechanism is configured to force the binaurals 15 apart when the first contact member is attached to at least the first binaural and when a portion of the first contact member 110 is forced: (I) towards the second contact member 115; (II) away from the second contact member; (III) towards the binaural fulcrum 45; (IV) away from the binaural fulcrum; and/or (V) towards the second binaural 15b.

In order to provide a better understanding of the manner in which the binaural spreading mechanism functions, the following disclosure is broken into several sections (namely sections I-V). These sections are each further subdivided into subsections to address several non-limiting subsets of the embodiments of the binaural spreading mechanism. The utilization of these sections and subsections is for the convenience of the reader only and is not to be construed as being limiting in any sense.

I. In embodiments in which the binaural spreading mechanism 100 comprises the first 110 and second 115 contact members, and in which the spreading mechanism is configured to force the binaurals apart as a portion of the contact members are forced towards each other, the spreading mechanism can comprise any suitable component or characteristic. In some non-limiting examples, the spreading mechanism comprises a (A) first handle and a second handle that are connected together (directly or indirectly) by the binaural fulcrum, (B) a sliding arm that increases in length as the contact members move towards each other, (C) two pairs of pivoting arms that force the binaurals apart as the arms straighten, and/or any other suitable component that is configured to force the binaurals apart when a portion of the first contact member and portion of the second contact member are moved towards each other. To better explain the spreading mechanism, these non-limiting subsets (A through C) are individually described below.

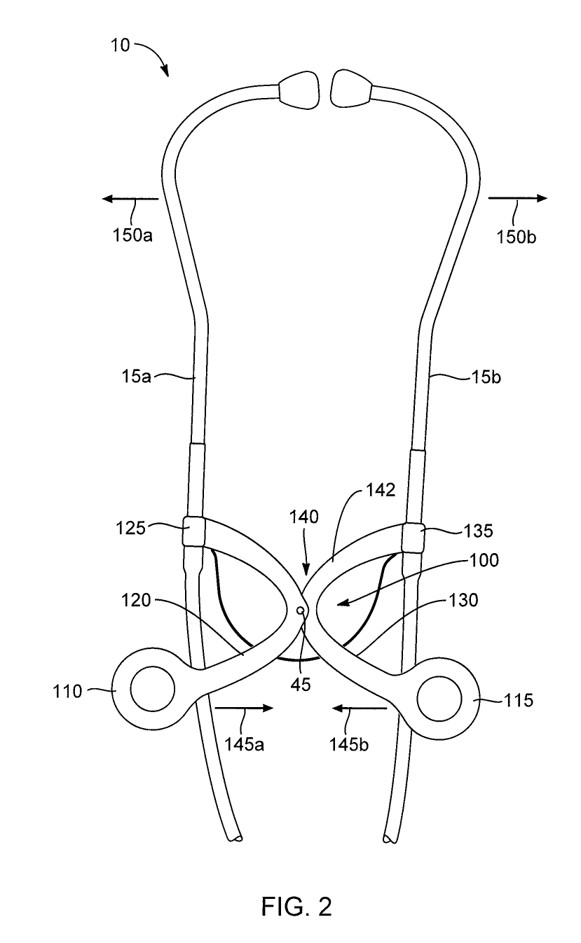

A. Where the spreading mechanism 100 comprises a first handle and a second handle, the first and second handles can have any suitable component. In one non-limiting example, FIGS. 1 and 2 show non-limiting embodiments in which the first handle 120 includes the first contact member 110 and a first connector 125 that is configured to connect to the first binaural 15a. Similarly, FIGS. 1 and 2 show non-limiting embodiments in which the second handle 130 comprises the second contact member 115 and a second connector 135 that is configured to connect to the second binaural 15b. In another non-limiting example, while the first contact member 110 can be directly attached to the first connector 125 and the second contact member 115 can be directly attached to the second connector 135, FIG. 1 shows an embodiment in which the first handle 120 and the second handle 130 each comprises a neck 137 that separate the contact members from their corresponding connectors.

While the first 120 and the second 130 handles can connect to the binaurals 15 in any suitable manner, FIGS. 1 and 2 show non-limiting embodiments in which the first connector 125 and the first contact member 110 both attach to the first binaural 15a on a first side of a center point 140 of the binaural fulcrum 45 (e.g., the leaf spring 48 in FIG. 1 and the pivot joint 142 in FIG. 2). Similarly, FIGS. 1 and 2 show embodiments in which the second connector 135 and the second contact member 115 both attach to the second binaural 15b on a second side of the fulcrum's center point 140. Accordingly, FIGS. 1 and 2 show that as the first 110 and second 115 contact members are forced towards each other, in the direction of arrows 145a and 145b, the first 15a and second 15b binaurals are forced away from each other, in the direction of arrows 150a and 150b. Thus, in the embodiments illustrated in FIGS. 1 and 2, the user can easily use a single hand to squeeze the first and second contact members together to spread the ear pieces apart and to thereby allow the stethoscope to be donned or removed from the user's ears.

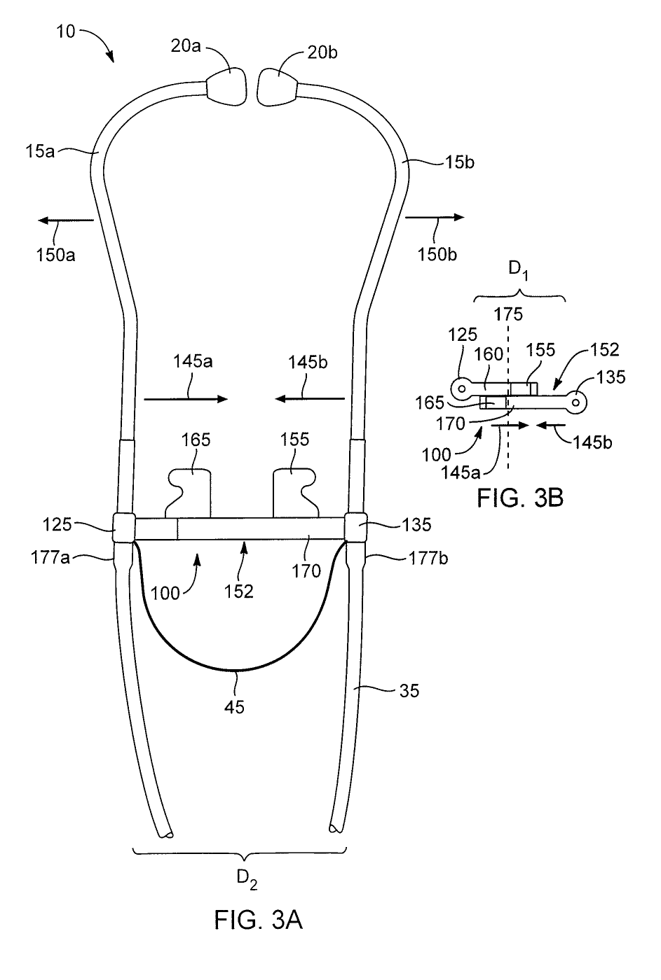

B. Where the spreading mechanism 100 comprises a sliding arm that includes the first contact member and the second contact member, and where the sliding arm increases in length as the two contact members move towards each other, the sliding arm can have any characteristic that allows it function as intended. In one non-limiting example, FIGS. 3A and 3B show different views of an embodiment in which the sliding arm 152 has the first contact member 155 (e.g., a finger hold) attached to a first slide member 160 and further has the second contact member 165 attached to a second slide member 170.

In another non-limiting example, FIGS. 3A and 3B show that the first slide member 152 and the second slide member 170 comprise the first connector 125 and the second connector 135, for attaching the sliding arm to the first 15a and second 15b binaurals, respectively.

In still another non-limiting example, FIGS. 3A and 3B show that the first slide member 160 and the second slide member 170 are slidably connected to each other. In this example, the first and second slide member can be slidably connected to each other in any suitable manner, including without limitation, through the use of a tongue on one slide member that is shaped to be slidably received by a groove in the other slide member.

In yet another non-limiting example, the first contact member 155 and the second contact member 165 can be disposed on the first slide member 160 and the second slide member 170 in any suitable manner that allows the distance D1 between the first 125 and the second 135 connectors to increase as the two contact members (e.g., 155 and 165) are forced towards each other. By way of non-limiting example, FIG. 3B shows an embodiment in which at least a portion of the first contact member 155 is disposed on a second side and at least a portion of the second contact member 165 is disposed on a first side of the slide arm's center point 175, when the binaurals 15 are in a closed position. As used herein, the term closed position may refer to any suitable position which the first 20a and the second 20b ear pieces are in close proximity to each other (as shown in FIG. 3A).

While the distance D1 between the first 125 and the second 135 connectors on the sliding arm 152 can be any suitable distance when the binaurals 15 are in the closed position, the sliding arm can be configured to increase the distance D1 between the first and second connectors to any suitable amount when the first and second connectors are forced towards each other. In some non-limiting embodiments, when the first 155 and the second 165 contact members are forced towards each other, the distance D1 between the connectors (e.g., 125 and 135) is as little as a length selected from about 5 millimeters (mm), about 1 centimeter (cm), and about 2 cm longer than the distance D2 between the binaurals' distal-most ends 177 (enclosed within the tubing 35 in FIG. 3A) when the binaurals are in the closed position. Moreover, in some non-limiting embodiments, as the first 155 and the second 165 contact members are forced towards each other, the distance D1 between the connectors is able to be extended to be as much as a length selected from about 2.5 cm, about 4 cm, about 7 cm, and about 10 cm longer than the distance D2 (when the binaurals are in the closed position) between the binaurals' distal-most ends 177.

Thus, FIGS. 3A and 3B shows that when the first 155 and the second 165 contact members are forced towards each other (e.g., through the use of a finger and thumb on the same hand) in the direction of arrows 145a and 145b, the length of the sliding arm 152 increases and the ear pieces 20 are forced apart in the direction of arrows 150a and 150b.

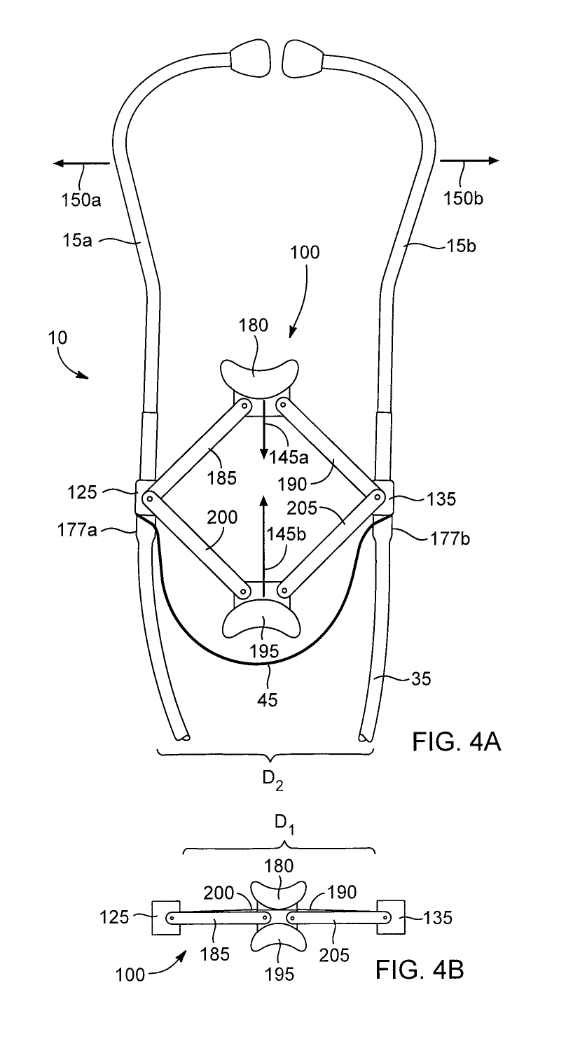

C. Where the binaural spreading mechanism 100 comprises two contact members and two pairs of pivoting arms that force the binaurals 15 apart as the contact members are forced towards each other, the spreading mechanism can include any suitable component or characteristic. In one non-limiting example, FIG. 4A shows the first contact member 180 is pivotally connected to a first 185 and a second 190 pivoting arm, which are respectively pivotally connected to the first connector 125 and the second connector 135. Similarly, in another non-limiting example, FIG. 4A shows that the second contact member 195 is pivotally connected to a third 200 and a fourth 205 pivoting arm, which are also pivotally connected to the first connector 125 and the second connector 135, respectively.

Where the pivoting arms (185, 190, 200, and 205) are pivotally attached to the first contact member 180, the second contact member 195, the first connector 125, and/or the second connector 135, the various components can be pivotally attached to each other in any suitable way, including without limitation, through the use of a pivot joint, a flexible material, or any other suitable pivotal connection.

Where the binaural spreading mechanism 100 comprises two contact members (e.g., 180 and 195) and two pairs of pivoting arms that force the binaurals 15 apart as the contact members are forced towards each other (and the pivoting arms are forced to straighten), the pivoting arms can be configured to separate the first 125 and second 125 connectors to be any suitable amount longer than the distance D2 between the distal-most ends 177 of the binaurals 15 when the binaurals are in the closed position. FIG. 4B illustrates that, in at least some non-limiting embodiments, when the first 180 and the second 195 contact members are forced towards each other, the distance D1 between the connectors (e.g., 125 and 135) is able to increase as little as a length selected from about 5 mm, about 1 cm, and about 2 cm longer than the distance D2 between the binaurals' distal-most ends 177 (enclosed within the tubing 35 in FIG. 4A) when the binaurals 15 are in the closed position. Moreover, in some non-limiting embodiments, FIG. 4B shows that when the first 180 and the second 195 contact members are forced towards each other, the distance D1 between the connectors (e.g., 125 and 135) is able to be extended to be as much as a length selected from about 2.5 cm, about 4 cm, about 7 cm, and about 10 cm longer than the distance D2 (when the binaurals are in the closed position) between the binaurals' distal-most ends 177.

Accordingly, FIGS. 4A and 4B show that where the user presses the first 180 and the second 195 contact members towards each other (in the direction of arrows 145a and 145b), the spreading mechanism 100 forces the first binaural 15a and the second binaural 15b away from each other, in the direction of arrows 150a and 150b.

II. In embodiments in which the one-handed spreading mechanism 100 is configured to force the binaurals 15 apart as a portion of the first contact member is forced towards the second contact member, the spreading mechanism can comprise any suitable component or characteristic. In some non-limiting examples, the spreading mechanism comprises (A) a pair of contact members that are attached to the binaurals; (B) a sliding arm that increases in length as the contact members move away from each other; and/or any other suitable component that acts to force the binaurals away from each other as a portion of its contact members are forced away from each other.

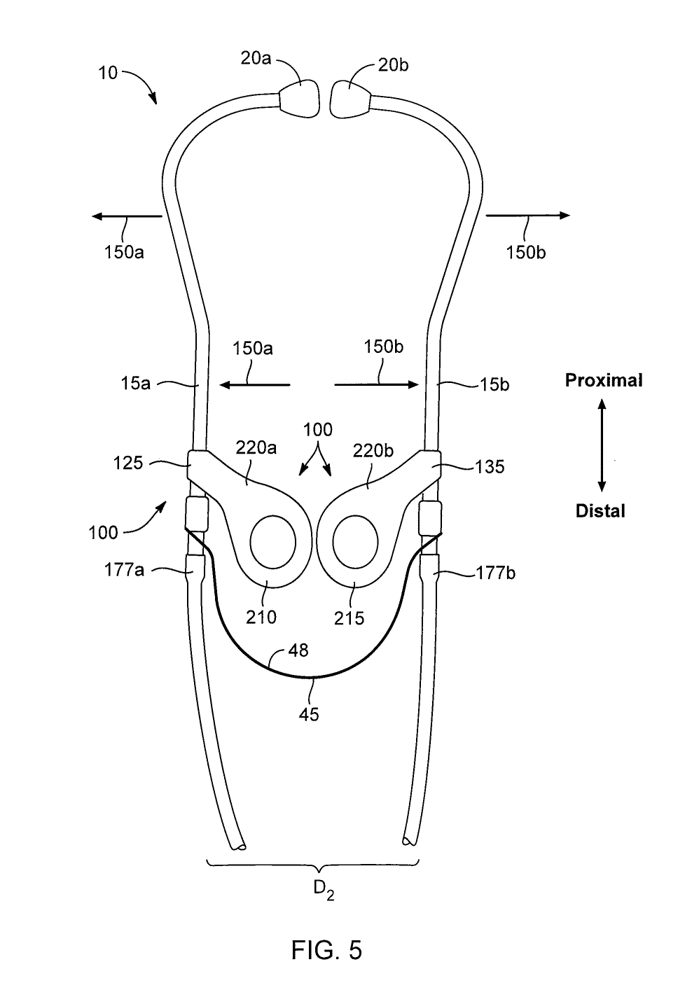

A. Where the spreading mechanism 100 comprises a pair of contact members that are attached to the binaurals 15, the spreading mechanism can comprise any suitable component or characteristic. In one non-limiting example, FIG. 5 shows an embodiment in which the spreading mechanism 100 comprises a first contact member 210 and a second contact member 215, which are attached to the first binaural 15a and to the second binaural 15b, respectively. In another non-limiting example, FIG. 5 shows the first 210 and second 215 contact members optionally comprise a loop. In still another non-limiting example, FIG. 5 shows that a neck 220 is optionally disposed between each contact member (e.g., 210 and 215) and a corresponding connector (e.g., 125 and 135). In yet another non-limiting example, FIG. 5 shows that the neck 220 optionally slopes distally so as to provide more room for the user's head and chin (not shown) between the ear pieces 20 and the binaural fulcrum 45.

As in all of the other embodiments described herein, the first 220a and/or second 220b contact members of the embodiments discussed in this subsection II(A) can attach to the first 15a and/or second 15b binaurals in any suitable manner. In some non-limiting examples, the first 220a and/or second 220b contact members that are described in this subsection II(A) connect to the first binaural 15a and/or second binaural 15b, respectively, by attaching to the binaurals 15, proximal to a portion of the binaural fulcrum 45; by attaching to the stethoscope on the binaural fulcrum 45 (e.g., connected to the leaf spring 48); by attaching to a clamp that connects the fulcrum to a binaural; and/or in any other suitable location. For instance, FIG. 5 shows a non-limiting example in which the first 210 and second 215 contact members are attached to the first binaural 15a and second binaural 15b, proximal to the binaural fulcrum 45 (e.g., leaf spring 48).

While the binaural spreading mechanism in FIG. 5 can function in any suitable manner, that figure shows that as the first 210 and second 215 contact members are forced apart (e.g., with the user's thumb and another finger on the same hand), the first 15a and second 15b binaurals are also forced apart, in the direction of arrows 150a and 150b.

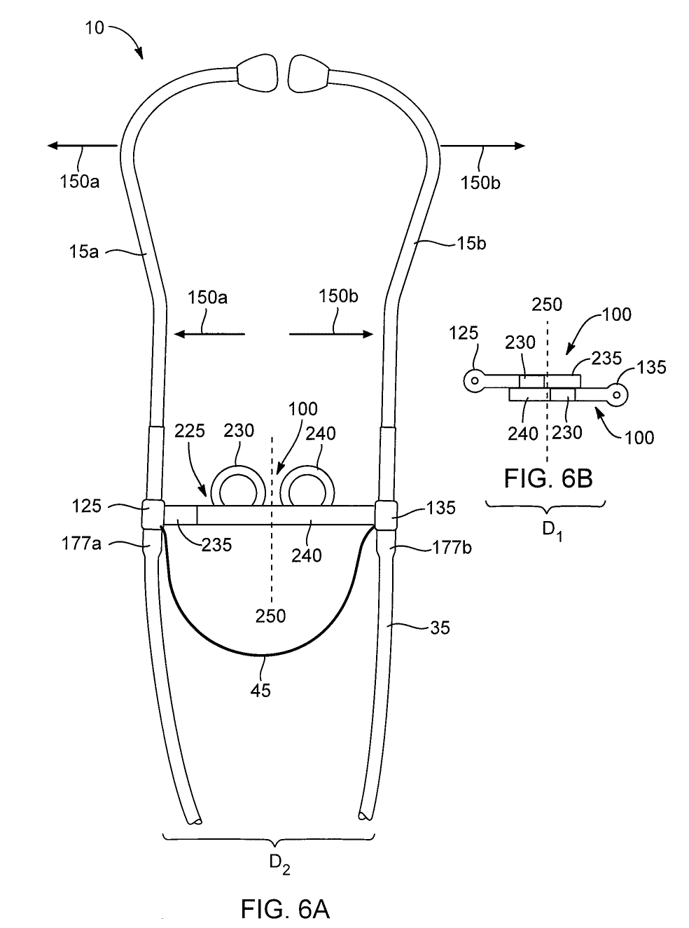

B. Where the binaural spreading mechanism 100 comprises a sliding arm that increases in length as its first and second contact members move away from each other, the spreading mechanism can comprise any suitable component or characteristic. By way of non-limiting example, FIGS. 6A and 6B depict different views of a spreading mechanism 100 comprising a sliding arm 225 that has a first contact member 230 disposed on a first slide member 235, and a second contact member 240 disposed on a second slide member 245. Additionally, FIGS. 6A and 6B illustrate that the first 235 and second 245 slide members are slidably connected to each other (e.g., via a tongue and groove system).

While the first 230 and second 240 contact members can be disposed on the sliding arm 225 in any suitable position, FIGS. 6A and 6B show that, in at least one non-limiting embodiment, at least a portion of the first contact member 230 is disposed on a first side of the slide arm's center point 250 and at least a portion of the second contact member 240 is disposed on a second side of the slide arm's center point 250, when the binaurals 15 are in the closed position.

While the distance D1 between the first 230 and the second 240 connectors on the sliding arm 225 can be any suitable distance when the binaurals 15 are in the closed position, the sliding arm can be configured to increase the distance D1 between the first 125 and second 135 connectors to any suitable amount when the first and second contact members are forced towards each other. In some non-limiting embodiments, when the first 230 and the second 240 contact members are forced towards each other, the distance D1 between the connectors (e.g., 125 and 135) is as little as a length selected from about 5 mm, about 1 cm, and about 2 cm longer than the distance D2 between the binaurals' distal-most ends 177 (enclosed within the tubing 35 in FIG. 6A) when the binaurals 15 are in the closed position. Moreover, in some non-limiting embodiments, as the first 230 and the second 240 contact members are forced towards each other, the distance D1 between the connectors is able to be extended to be as much as a length selected from about 2.5 cm, about 4 cm, about 7 cm, and about 10 cm longer than the distance D2 (when the binaurals are in the closed position) between the binaurals' distal-most ends 177.

Thus, FIGS. 6A and 6B show that where the first 230 and second 240 contact members are moved away from each other, in the direction of arrows 150a and 150b, the first 125 and second 135 connectors are forced further apart. In this manner, when the first 125 and the second 135 connectors are attached to the first 15a and second 15b binaurals, respectively, the user can easily spread the binaurals apart by spreading the first 230 and second 240 contact members apart (e.g., with the user's thumb and an opposing finger).

III. In embodiments in which the one-handed spreading mechanism 100 comprises a first contact member, and the spreading mechanism is configured to force the binaurals 15 apart as a portion of the contact member is forced towards a portion of the binaural fulcrum 45 (e.g., leaf spring 48), the spreading mechanism can comprise any suitable component or characteristic. In some non-limiting examples, the spreading mechanism comprises (A) a pivoting contact member that is longer than a distance between the distal-most end of the two binaurals, (B) a contact member that is configured to force the two binaurals apart as the contact member slides distally on the two binaurals, a bendable (e.g., (C) a pair of pivoting arms and/or (D) a flexible member) that is configured to force the binaurals apart as it is straightened, and/or any other suitable component that causes the binaurals to move away from each other as a portion of the first contact member is forced towards the binaural fulcrum.

A. Where the spreading mechanism 100 comprises a pivoting contact member that is longer than a distance D2 between the distal-most ends of the binaurals 15 when the binaurals are in the closed position, the spreading mechanism can comprise any suitable component or characteristic.

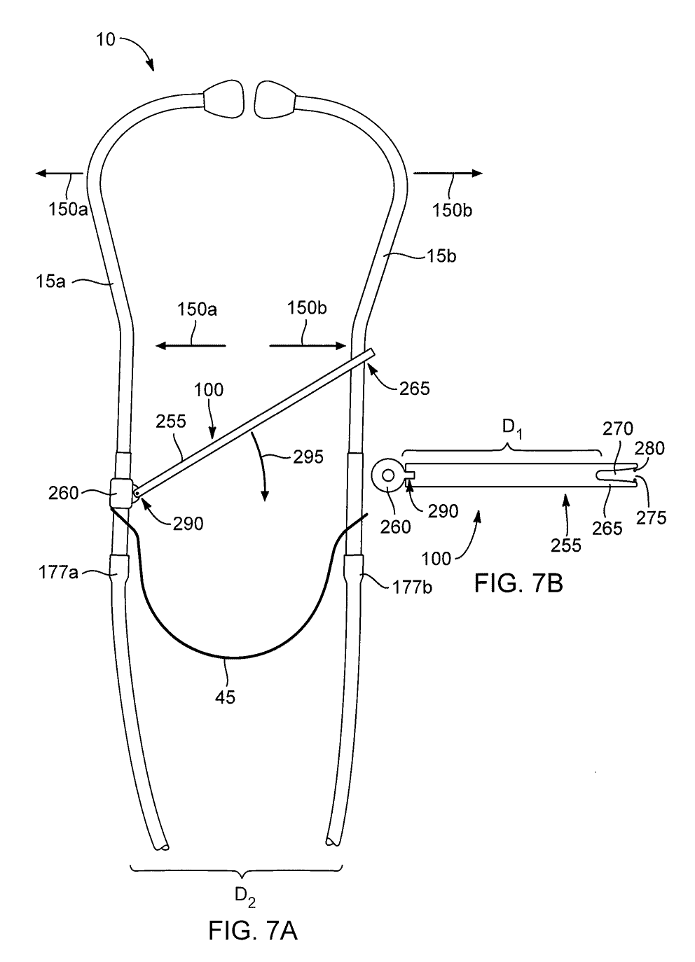

In one non-limiting example, FIG. 7A shows the pivoting contact member 255 comprises a first connector 260 that is configured to pivotally connect the contact member 255 to the first binaural 15a). In another non-limiting example, FIGS. 7A and 7B shows the contact member 255 further comprises a second connector 265 that is configured to slide proximally and distally on the second binaural 15b. In this latter example, the second connector can comprise any suitable characteristic that allows it to slide distally and proximally on the second binaural. By way of non-limiting example, the second connector 255 can comprise an elongated slot (as shown by 270 in FIG. 7B), have a closed end, have an open end (as shown by 275 in FIG. 7B), comprise one or more binaural catches (such as those shown as 280 in FIG. 7B), and/or have any other suitable characteristic that allows the spreading mechanism to function as intended.

In another non-limiting example, where the spreading mechanism 100 comprises the pivoting contact member 255, the distance D1 between the first connector 260 and the second connector 265 can be any suitable length longer than the distance D2 between the distal-most ends 177 of the first 15a and second 15b binaurals when the binaurals are in the closed position. In some non-limiting embodiments, the distance D1 between the connectors is as little as a length selected from about 5 mm, about 1 cm, and about 2 cm longer than the distance D2 between the binaurals' distal-most ends 285 (when the binaurals are in the closed position). Moreover, in some non-limiting embodiments, distance D1 between the connectors 260 and 265 is as much as a length selected from about 2.5 cm, about 4 cm, about 7 cm, and about 10 cm longer than the distance D2 between the binaurals' distal-most ends 285 (when the binaurals are in the closed position).

In still another non-limiting example, the pivoting contact member 255 is pivotally connected to the first connector 260 in any suitable manner, including, without limitation, through a pivot joint (as shown at 290 in FIG. 7A) and/or with a flexible material (not shown).

Thus, as described above, as a portion of the pivoting contact member 255 is forced towards the binaural fulcrum 45, in the direction of arrow 295, the first 15a and second 15b binaurals are forced apart, in the direction of arrows 150a and 150b.

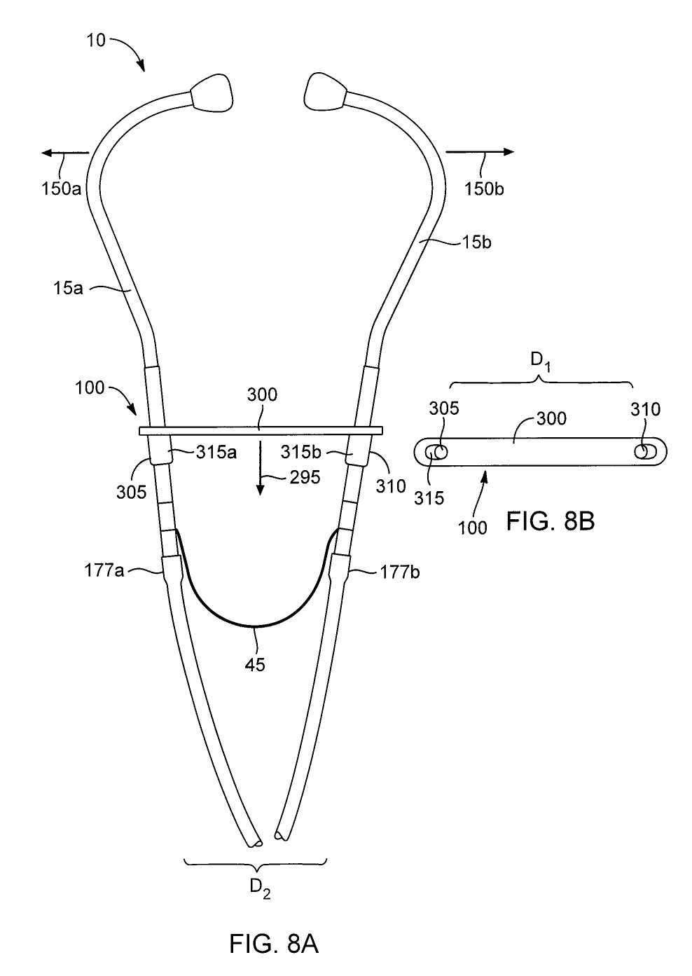

B. Where the spreading mechanism 100 comprises a first contact member that is configured to force the binaurals 15 apart as the contact member slides distally across the binaurals, the spreading mechanism can comprise any suitable component or characteristic. By way of non-limiting example, FIG. 8A shows an embodiment in which the first contact member 300 comprises a first connector 305 and a second connector 310 that are configured to slide distally and proximally on the first 15a and second 15b binaurals, respectively. In this example, the first and second connectors can have any suitable characteristic that allows the spreading mechanism to function as intended. FIG. 8A shows a non-limiting embodiment in which the first 305 and second 310 connectors each comprises a sleeve 315 that is configured to prevent the contact member 300 from binding on the binaurals 15 as the contact member is forced distally on the binaurals.

In another non-limiting example, when the binaurals 15 are in the closed position, the distance D1 between the first 305 and second 310 connectors can be any suitable distance longer than the distance D2 (as shown in FIG. 7A) between the distal-most ends 177 of the binaurals. In some non-limiting embodiments, the distance Dl between the connectors is as little as a length selected from about 5 mm, about 1 cm, and about 2 cm longer than the distance D2 between the binaurals' distal-most ends 285 (when the binaurals are in the closed position). Furthermore, in some non-limiting embodiments, when the binaurals are in the closed position, the distance D1 between the connectors 305 and 310 is as much as a length selected from about 2.5 cm, about 4 cm, about 7 cm, and about 10 cm longer than the distance D2 between the binaurals' distal-most ends 177.

Thus, as a portion of the first contact member 300 is forced distally on the binaurals (e.g., towards the binaural fulcrum 45 and in the direction of arrow 295), the first 15a and second 15b binaurals are forced apart, in the direction of arrows 150a and 150b.

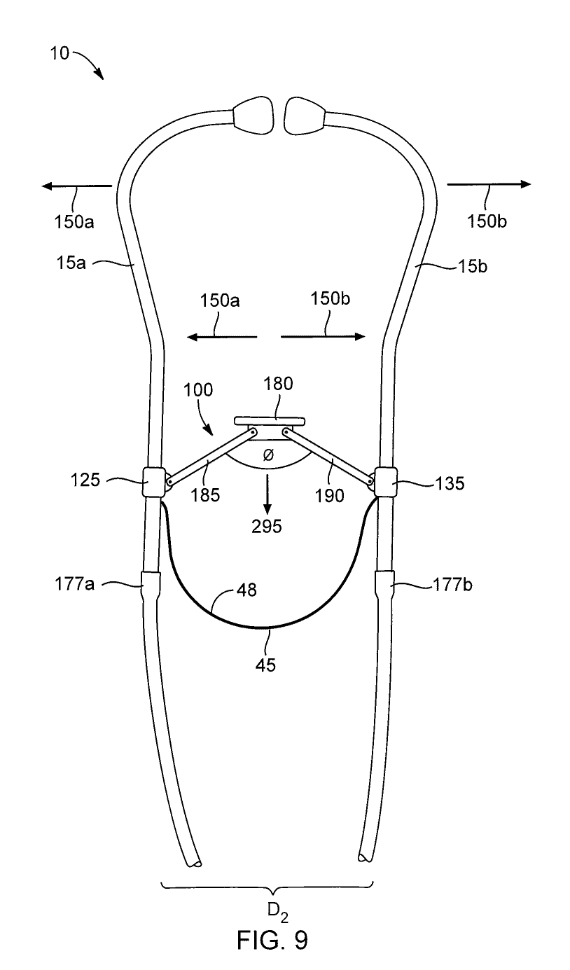

C. Where the spreading mechanism 100 comprises a first contact member having a pair of pivoting arms that are configured to force the binaurals 15 apart as the contact member is forced towards the fulcrum 45, the spreading mechanism can comprise any suitable component or characteristic. By way of non-limiting example, FIG. 9 illustrates an embodiment in which the spreading mechanism 100 comprises the first contact member 180, the first pivoting arm 185, and the second pivoting arm 190 that are discussed above in section I(C).

Where the spreading mechanism 100 comprises the first contact member 180, the first pivoting arm 185, and the second pivoting arm 190, and where the spreading mechanism separates the binaurals 15 as a portion of the first contact member is forced towards the fulcrum 45, the first contact member can be connected to the stethoscope in any suitable manner that allows it to function as described. In one non-limiting example, FIG. 9 shows the first contact member 180 is attached to the stethoscope 10 so as to spread the binaurals 15 apart when the contact member 180 is forced distally (in the direction of arrow 295) toward the fulcrum 45. In another non-limiting example (not illustrated), the first contact member 180 is attached so that its two pivoting arms 185 and 190 force the binaurals 15 apart as the contact member 180 is forced towards the fulcrum 45 at any other suitable angle (e.g., towards or away from the user when the user is donning the stethoscope).

D. Where the spreading mechanism 100 comprises a flexible contact member that is configured to force the binaurals 15 apart as a portion of the flexible member is forced towards the fulcrum 45 and the flexible contact member is thereby forced to straighten, the spreading mechanism can comprise any suitable component or characteristic. In one non-limiting example, the flexible contact member comprises a strip of material that is able to bow under the tension of a binaural bias (e.g., leaf spring 48) and which is capable of becoming less bowed (e.g., straightened) as the user applies pressure to the convex side of the contact member.

In another non-limiting example, while some embodiments of the flexible contact member comprise a pliable material that is not resilient, in other embodiments, the flexible contact member comprises a resilient piece of material (such as a leaf spring).

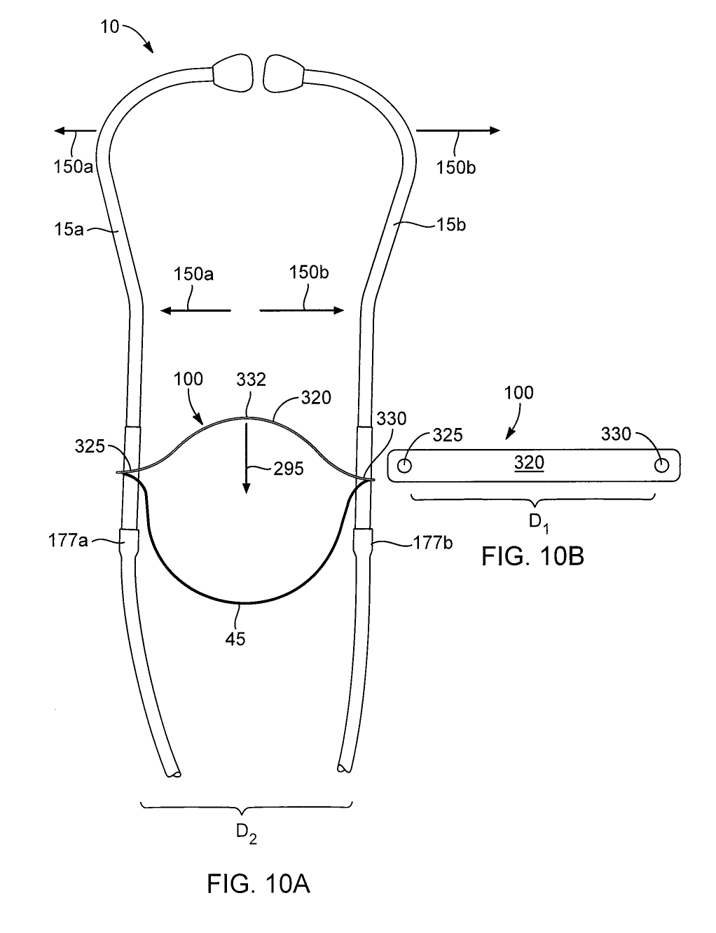

In another non-limiting example, FIGS. 10A and 10B show that the flexible contact member 320 comprises a first connector 325 and a second connector 330. In this example, the distance D1 between the first 325 and second 330 connectors can be any suitable distance that allows the contact member to function as intended. Specifically, FIGS. 10A and 10B show that when the binaurals 15 are in the closed position, the distance D1 between the first 325 and second 330 connectors is longer than the distance D2 between the binaurals' distal-most ends 1775. Accordingly, FIG. 10A shows that when the binaurals 15 are in the closed position, the flexible contact member 320 is caused to buckle. Thus, pressure is applied to the convex 332 side of the contact member in the direction of arrow 295, the contact member is caused to straighten and the distance D1 between the first 325 and the second 330 connectors increases to cause the binaurals to be forced apart.

When the flexible contact member is fully straightened, the distance D1 between the first 325 and the second 330 connectors may be any suitable length longer than the distance D2 between the distal-most ends 177 of the binaurals (when the binaurals are in the closed position). In some non-limiting embodiments, the distance D1 between the connectors is as little as a length selected from about 5 mm, about 1 cm, and about 2 cm longer than the distance D2 between the binaurals' distal-most ends 285 (when the binaurals are in the closed position). Furthermore, in some non-limiting embodiments, when the binaurals are in the closed position, the distance D1 between the first 325 and second 330 connectors is as much as a length selected from about 2.5 cm, about 4 cm, about 7 cm, and about 10 cm longer than the distance D2 between the binaurals' distal-most ends 177 (when the binaurals are in the closed position).

Where the spreading mechanism comprises the flexible contact 320 member and the contact member is configured to attach to the stethoscope so as to separate the binaurals when a portion of the contact member is forced towards the binaural fulcrum, the spreading mechanism can attach to the stethoscope so as to be forced towards the fulcrum at any suitable angle. By way of non-limiting illustration, FIG. 10A shows an embodiment in which the bendable contact member 320 attaches to the stethoscope 10 so as to force the binaurals 15 apart as the contact member 320 is forced towards the fulcrum in the direction of arrow 295. In other non-limiting embodiments, however, the bendable contact member 320 attaches to the stethoscope 10 so as to force the binaurals apart when the user forces a portion of the contact member towards the fulcrum 45 at any other suitable angle (e.g., towards or away from the user, when the user is donning the stethoscope).

IV. In embodiments in which the one-handed spreading mechanism 100 comprises the first contact member, and in which the spreading mechanism is configured to force the binaurals apart as the contact member is forced away from the binaural fulcrum 45, the spreading mechanism can comprise any suitable component or characteristic. By way of non-limiting example, the binaural spreading mechanism can comprise a bendable contact member (such as (A) a pair of pivoting arms or (B) a flexible member) that forces the binaurals apart as the bendable member is straightened and/or any other suitable component that is capable of forcing the binaurals apart as a portion of the component is forced away from the binaural fulcrum.

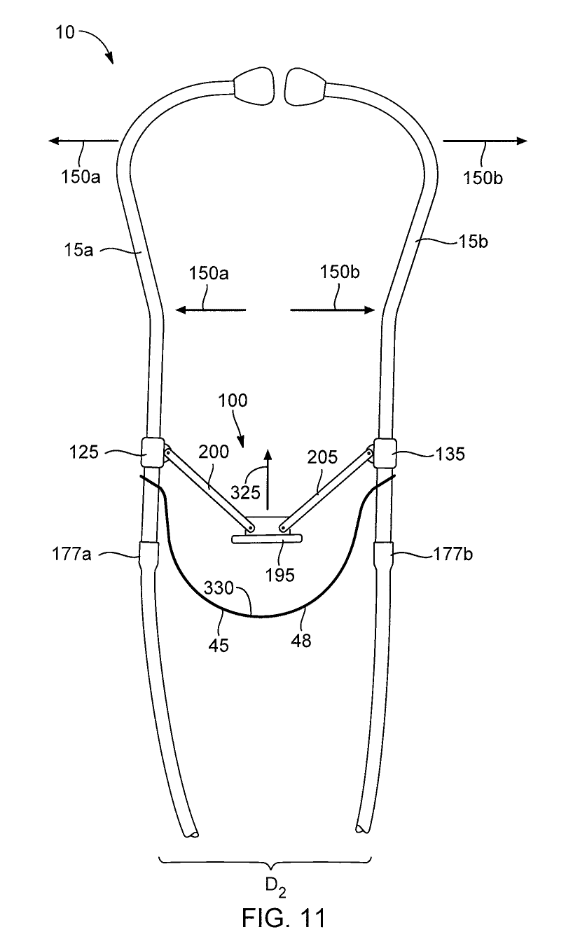

A. Where the spreading mechanism 100 comprises a contact member having a pair of pivoting arms that are configured to force the binaurals 15 apart as a portion of the contact member is forced away from the fulcrum 45, the spreading mechanism can comprise any suitable component or characteristic. By way of non-limiting example, FIG. 11 illustrates an embodiment in which the contact member comprises the second contact member 195, the third pivoting arm 200, and the fourth pivoting arm 205 that are discussed above in section I(C).

In this embodiment, the second contact member 195 and the third 200 and forth 205 pivoting arms can connect to the stethoscope 10 in any suitable manner that allows the pivoting arms to force the binaurals 15 apart as the a portion of the contact member is forced away from the fulcrum 45. By way of illustration, FIG. 11 shows a non-limiting embodiment in which the third 200 and fourth 205 pivoting arms are configured to force the binaurals 15 apart as the second contact member 195 is moved proximally, in the direction of arrow 325. In another non-limiting example (not shown), the second contact member 195 attaches to the stethoscope so as to force the binaurals apart when the contact member is forced away from the fulcrum at any other suitable angle (e.g., towards or away from the user, when the user is donning the stethoscope).

While the embodiments described in this subsection can be used in any suitable manner, in some instances, the user forces the second contact member 195 to move proximally by applying pressure to the concave portion 330 of the fulcrum 45 (e.g., leaf spring 48) with one or more digits, and then applying pressure in the opposite direction against the second contact member 195 with another digit (e.g., the user's thumb) on the same hand.

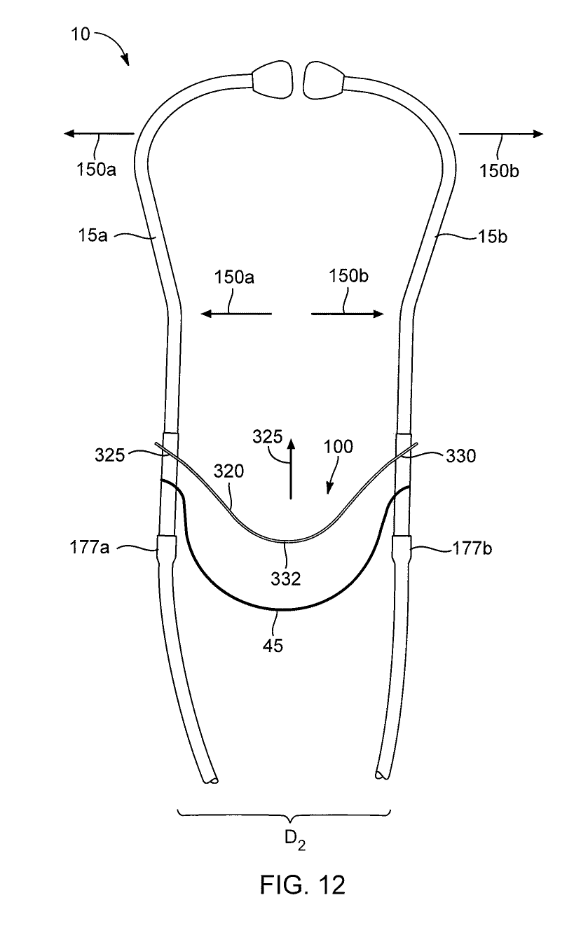

B. Where the spreading mechanism 100 comprises a flexible member that forces the binaurals 15 apart as a portion of the flexible member is forced away from the fulcrum, the spreading mechanism can comprise any suitable component or characteristic. By way of non-limiting example, FIG. 12 shows an embodiment in which the binaural spreading mechanism 100 comprises the bendable contact member 320 that is discussed above in Section III(D). In contrast with the embodiment shown in FIG. 10A, however, FIG. 12 shows a non-limiting embodiment in which the bendable contact member 320 is connected to the stethoscope 10 in a manner that allows the user to separate the binaurals 15 by forcing a convex portion 332 of the contact member 320 away from the fulcrum 45 (e.g., leaf spring 48).

While FIG. 12 shows a non-limiting embodiment in which the bendable contact member 320 forces the binaurals 15 apart as a portion of the bendable member is forced in the direction of arrow 325, the bendable member can be attached to the stethoscope so as to force the binaurals apart when the member is forced in any other direction that allows a portion of the bendable member to move away from the fulcrum. In one non-limiting example, the bendable member is configured to spread the binaurals apart as the convex portion 332 is forced away from the fulcrum 45 and towards or away from the user, as the user dons the stethoscope.

V. In embodiments in which the binaural spreading mechanism 100 is configured to force the binaurals 15 apart as a portion of the first contact member is moved towards the second binaural 15b, the spreading mechanism can comprise any suitable component or characteristic. In some non-limiting embodiments, the spreading mechanism comprises (A) a first contact member that is configured to connect to the first binaural so as to extend laterally past the second binaural when the binaurals are in the closed position; (B) a first contact member that comprises a first portion, which is configured to connect to the first binaural, and a hand-contact portion, which is configured to connect to the second binaural; and/or any other suitable mechanism that separates the binaurals as a portion of the first contact moves towards the second binaural.

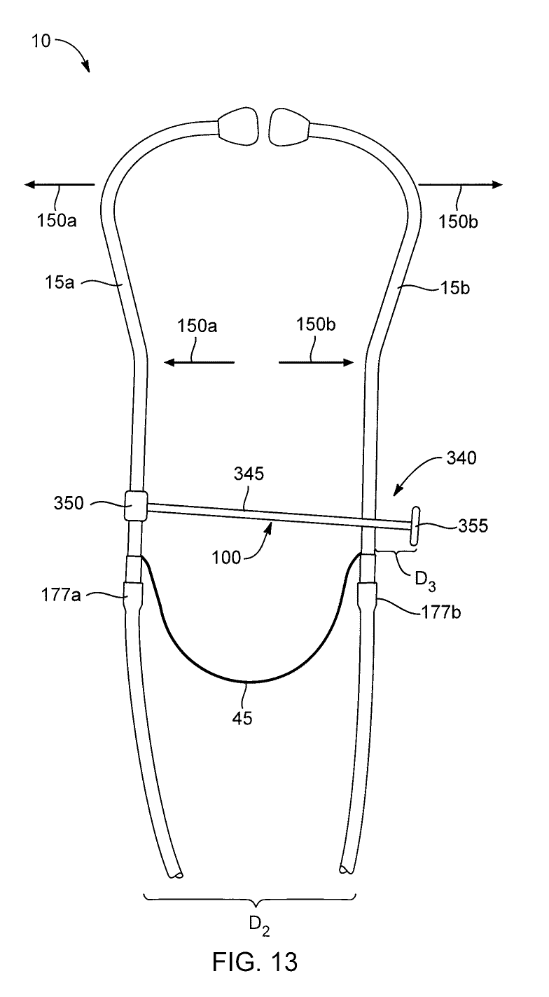

A. Where the spreading mechanism 100 comprises a first contact member that connects to the first binaural 15a and extends laterally past the second binaural 15b (when the binaurals are in the closed position), the spreading mechanism can comprise any suitable component or characteristic. In one non-limiting example, FIG. 13 shows an embodiment in which the first contact member 340 comprises a shaft 345 that connects to the first binaural 15a (e.g., via the first connector 350) and that comprises a hand-contact portion 355, which extends laterally past the second binaural 15b when the binaurals are in the closed position.

In another non-limiting example, when the binaurals 15 are in the closed position, the first contact member 340 can extend any suitable distance past the second binaural 15b. FIG. 13 shows that, in some non-limiting embodiments, when the binaurals 15 are in a closed position, the hand-contact portion 355 of the first contact member 340 extends laterally past the second binaural 15b by a distance D3 that is selected from a length that is as little as about 5 mm, about 1 cm, and about 2 cm. In other embodiments, when the binaurals are in the closed position, the hand-contact portion 355 of the first contact member 340 extends laterally past the second binaural 15b by a distance D3 that is as much as about 2.5 cm, about 4 cm, about 7 cm, and about 10 cm.

In still another non-limiting example (not illustrated), the second binaural optionally comprises a catch or a channel that holds the shaft 345 next to the second binaural 15b as the contact portion 355 is forced towards the second binaural.

In yet another non-limiting example, the shaft 345 can connect to the first connector 350 in any suitable manner, including without limitation, by being integrally formed with, being pivotally attached to, and/or otherwise being connected to the first connector.

While the spreading mechanism comprising the first contact member 340 can function in any suitable manner, in some non-limiting embodiments, the mechanism allows the user to force the binaurals apart by holding the second binaural 15b with one hand and using a thumb or another digit on that same hand to push the hand-contact portion 355 of the first contact member 340 medially.

B. Where the spreading mechanism 100 comprises a first contact member that includes a first portion and a hand-contact portion, and wherein the mechanism is configured to spread the binaurals 15 apart as the hand-contact portion is forced towards the second binaural 15b, the spreading mechanism can comprise any suitable component or characteristic.

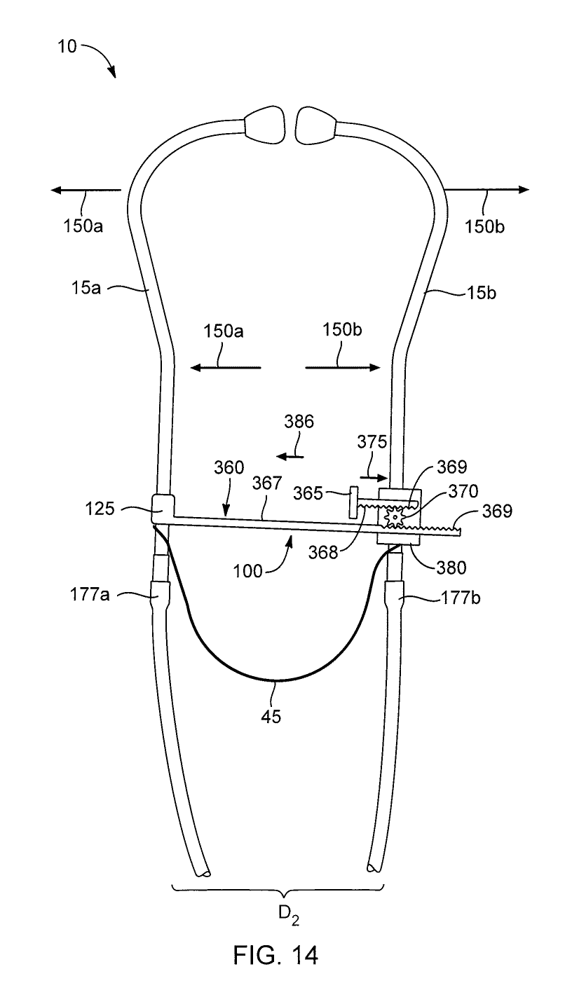

In one non-limiting example, the first portion and the hand-contact portion comprise separate components, which are mechanically and movably connected through any suitable number of wheels, gears, and/or other suitable devices that are configured to cause the distance between the binaurals to increase as the hand-contact portion is forced towards the second binaural. By way of illustration, FIG. 14 shows a non-limiting embodiment in which the first portion 360 and the hand-contact portion 365 of the spreading mechanism 100 each comprises a separate shaft (e.g., 367 and 368, respectively). Although, these shafts (367 and 368) can be mechanically and movably connected in any suitable manner that allows the spreading mechanism to function as intended, FIG. 14 shows a non-limiting embodiment in which a gear 370 articulates against teeth 369 on the shaft 367 of the first portion 360 and against teeth 369 on the hand-contact portion, to force the shafts to move in opposite directions.

In another non-limiting example, FIG. 14 shows that while the first portion's shaft 367 connects to the first binaural 15a through the first connector 125, the hand-contact portion (along with the gear 370 and the shaft 367 of the first portion) attach to the second binaural 15b through a housing 380 that comprises a second connector (not shown).

Thus, as shown in the non-limiting embodiment in FIG. 14, as the hand-contact portion 365 of the first contact member is forced towards the second binaural 15b (in the direction of arrow 375), the gear 370 forces the first portion to move in the direction of arrow 386 (with respect to the second binaural 15b) so that the first and second binaurals are cause to move away from each other. As a result, the user can easily don the embodiment of the stethoscope in FIG. 14 by pressing the hand-contact portion 365 towards the second binaural 15b.

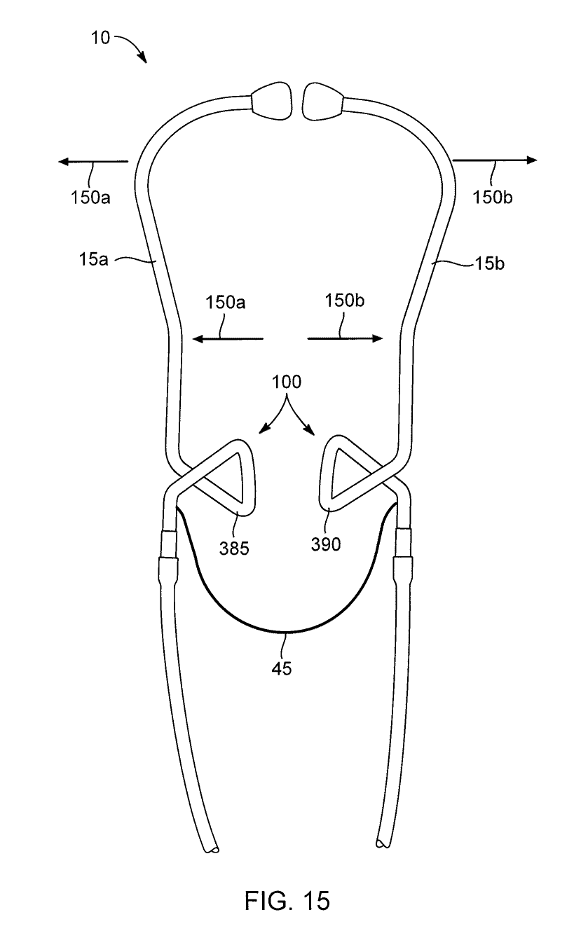

In addition to the non-limiting embodiments described above, the binaural spreading mechanism 100 can be varied in any suitable manner. For instance, while FIGS. 1 through 14 illustrate non-limiting embodiments in which the binaural spreading mechanism 100 is configured to be selectively connected to and/or selectively disconnected from the stethoscope 10 as a retrofit component, in some other non-limiting embodiments, the binaural spreading mechanism is integrally formed on the stethoscope.

Where the spreading mechanism 100 is integrally formed on the stethoscope 10, the spreading mechanism can be formed on any suitable portion of the stethoscope that allows the mechanism to perform its intended purpose. In one non-limiting example, FIG. 15 shows the binaural spreading mechanism 100 is formed by bending the first 15a and/or the second 15b binaurals to form the first 385 and/or second 390 contact members. While the contact members 385 and 390 in this example can have any suitable shape or other characteristic that allows the user to catch at least one digit in a contact member, FIG. 15 shows a non-limiting embodiment, in which the first 385 and second 390 contact members have a loop-shaped appearance.

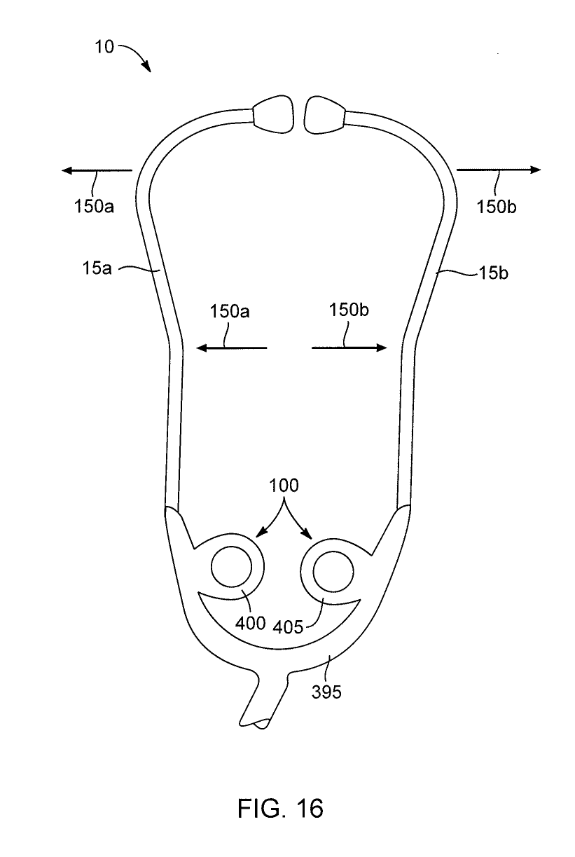

In another non-limiting example in which the binaural spreading mechanism 100 is formed on the stethoscope 10, the mechanism is formed on tubing that connects to the binaurals 15. For instance, FIG. 16 shows a non-limiting embodiment in which the binaurals 15 are connected by tubing 395 (which optionally comprises an internal leaf spring (not shown)). Moreover, FIG. 16 shows that the first 400 and/or second 405 contact members are formed directly on the tubing 395. While the contact members in this non-limiting example can have any suitable shape or other characteristic that allows the user to catch at least one digit in a contact member to force the binaurals apart, FIG. 16 shows a non-limiting embodiment in which the first 400 and second 405 contact members each have a loop-shaped appearance.

While the embodiments shown in FIGS. 15 and 16 can function in any suitable manner, those figures show that, in at least some non-limiting instances, as the first contact member (385 in FIGS. 15 and 400 in FIG. 400) is moved in the direction of arrow 150a, and as the second contact member (390 in FIG. 15 and 405 in FIG. 16) is moved in the direction of arrow 150b, the binaurals 15 are also forced to spread apart.

In another example of a manner in which the spreading mechanism 100 can be modified, in some non-limiting embodiments (not shown) the spreading mechanism comprises a biasing device that is configured to bias the binaurals 15 together. In such embodiments, the biasing device can comprise any suitable component, including without limitation, a coil spring, a spiral spring, a volute spring, an elastic member, and/or any other suitable component. Additionally, while the spreading mechanism can comprise a biasing device in addition to a leaf spring on the stethoscope, in some non-limiting embodiments, the biasing device on the spreading mechanism is used in place of the leaf spring.

The one-handed, binaural spreading mechanism 100 can be made of any suitable material. In one non-limiting example, the various components (i.e., the contact members, connectors, etc.) of the spreading mechanism are made of any suitable plastic, rubber, polymer, metal, ceramic, composite, wood, and/or other suitable substance.

The spreading mechanism 100 can also be made in any suitable manner that allows it to function as intended. By way of non-limiting example, the spreading mechanism can be formed through molding, extruding, cutting, bending, filing, bonding, etching, and/or any other suitable process.

As discussed above, the binaural spreading mechanism 100 can be attached (directly or indirectly) to one or both binaurals 15 through one or more connectors. These connectors, in turn, can attach to the stethoscope 10 in any suitable manner. Some examples of such attachment methods include, but are not limited to, clamping, chemically bonding, mechanically bonding, frictionally attaching, slidingly attaching, attaching with a fastener (e.g., screw, rivet, etc.), and/or otherwise attaching the spreading mechanism to a portion of the stethoscope (e.g., one or both binaurals 15, the leaf spring 48, the tubing 35, etc.).

Where a connector is attached to a binaural 15, the connector can be placed on the binaural in any suitable manner. In one non-limiting example, the ear piece 20 is removed from the proximal end of the binaural, the connector is slid over the binaural, and the ear piece is replaced. In another non-limiting example, the spreading mechanism is forced over a side of the binaural before being attached to the binaural. In still another non-limiting example, the spreading mechanism is simply attached to the side of the binaural (e.g., via chemical bonding and/or another suitable method.).

As mentioned, the described one-handed spreading mechanism offers several beneficial characteristics. In one non-limiting example, the spreading mechanism allows the user to don or remove the stethoscope with a single hand while the user's other hand can be used to perform other functions. In another non-limiting example, the spreading mechanism is easy to use, even during emergency situations. In still another non-limiting example, because some embodiments of the spreading mechanism are configured to be retrofit to stethoscopes, users can easily modify their current stethoscope.

Thus, as discussed herein, the embodiments of the present invention embrace systems and methods for donning a stethoscope on a user through the use of a single hand.

The present invention may be embodied in other specific forms without departing from its spirit or essential characteristics. The described embodiments and examples are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A one-handed, binaural spreading mechanism configured to be retrofitted on a stethoscope, the spreading mechanism comprising:
a first contact member connected to a first connector that is sized and shaped to attach the first contact member to a first binaural on the stethoscope, wherein the first contact member is sized and shaped to attach to the first binaural in such a manner that when the first contact member is attached to the first binaural, the first binaural is forced to move away from a second binaural when a portion of the first contact member is moved in an binaural spreading direction.

2. The binaural spreading mechanism of claim 1, wherein the binaural spreading direction is selected from:
a) towards a second contact member that is attached to the second binaural,
b) away from the second contact member that is attached to the second binaural,
c) towards a binaural fulcrum that connects the first binaural to the second binaural,
d) away from the fulcrum, and
e) towards the second binaural.

3. The binaural spreading mechanism of claim 2, wherein the first and second contact members are sized and shaped such that when the first and second contact members are respectively connected to the first and second binaurals so that the first and second contact members are disposed between the first and second binaurals, the first and second binaurals are forced to move away from each other when the first and second contact members are forced away from each other.

4. The binaural spreading mechanism of claim 2, further comprising:
a first handle that connects the first contact member to the first connector; and
a second handle that connects the second contact member to a second connector, which is sized and shaped to attach the second contact member to the second binaural, wherein the first contact member and the first connector are configured to attach to the first binaural on a first side of a center point of the fulcrum, and wherein the second contact member and the second connector are configured to attach to the second binaural on a second side of the center point.

5. The binaural spreading mechanism of claim 2, further comprising:
a first slide member comprising the first connector; and
a second slide member comprising a second connector that is sized and shaped to attach to the second binaural, wherein the first slide member is slidably attached to the second slide member, and wherein the first contact member is attached to the first slide member and the second contact member is attached to the second slide member such that a distance between the first connector and the second connector will increase when the first and second contact members are forced towards each other.

6. The binaural spreading mechanism of claim 2, further comprising:
a first slide member comprising the first connector; and
a second slide member comprising a second connector that is sized and shaped to attach to the second binaural, wherein the first slide member is slidably attached to the second slide member, and wherein the first contact member is attached to the first slide member and the second contact member is attached to the second slide member such that a distance between the first connector and the second connector will increase when the first and second contact members are forced away from each other.

7. The binaural spreading mechanism of claim 1, wherein the first contact member further comprises:
a second connector that is sized and shaped to attach to the second binaural, wherein the first connector is sized and shaped to pivotally connect the first contact member to the first binaural, wherein the second connector is sized and shaped to articulate proximally and distally on the second binaural, and wherein a distance between the first connector and the second connector is larger than a distance between a distal-most end of the first binaural and the second binaural.

8. The binaural spreading mechanism of claim 1, wherein the first contact member further comprises:
a second connector that is sized and shaped to connect the first contact member to the second binaural, wherein the first connector and the second connector are sized and shaped to slide distally and proximally on the first and second binaurals, respectively, and wherein a distance between the first connector and the second connector is larger than a distance between the distal-most ends of the first binaural and the second binaural.

9. The binaural spreading mechanism of claim 1, wherein the first contact member further comprises:
a second connector that is sized and shaped to attach the first contact member to the second binaural, wherein the spreading mechanism is bendable so that the spreading mechanism is able to extend and cause a distance between the first connector and the second connector to be longer than a distance between a distal-most end of the first binaural and the second binaural.

10. The binaural spreading mechanism of claim 2, further comprising a shaft that attaches a hand-contact portion of the first contact member to the first connector, wherein a distance between the hand-contact portion and the first connector is longer than a distance between the distal-most end of the first binaural and the distal-most end of the second binaural.

11. A stethoscope, comprising:
a first binaural;
a second binaural; and
a one-handed, binaural spreading mechanism that allows a user to force the first binaural away from the second binaural with the use of a single hand, wherein the binaural spreading mechanism comprises:
a first contact member that is attached to the first binaural, wherein the first contact member is sized and shaped such that when it is forced to move in a binaural spreading direction, the first and second binaurals are forced to move away from each other.

12. The stethoscope of claim 11, wherein the binaural spreading mechanism comprises a retrofit component that is selectively attached to the stethoscope.

13. The stethoscope of claim 11, wherein the binaural spreading mechanism is formed on the stethoscope.

14. The stethoscope of claim 11, wherein the binaural spreading direction is selected from:
a) towards a second contact member that is attached to the second binaural,
b) away from the second contact member,
c) towards a binaural fulcrum that connects the first binaural to the second binaural,
d) away from the fulcrum, and
e) towards the second binaural.

15. The stethoscope of claim 14, wherein the first and second contact members are sized and shaped such that when the first and second contact members are respectively connected to the first and second binaurals so that the first and second contact members are disposed between the first and second binaurals, the first and second binaurals are forced to move away from each other when the first and second contact members are forced away from each other.

16. The stethoscope of claim 14, further comprising:
a first handle that connects the first contact member to the first connector; and
a second handle that connects the second contact member to a second connector, which is sized and shaped to attach the second contact member to the second binaural, wherein the first contact member and the first connector are configured to attach to the first binaural on a first side of a center point of the fulcrum, and wherein the second contact member and the second connector are configured to attach to the second binaural on a second side of the center point.

17. A stethoscope, comprising:
a first binaural;
a second binaural; and
a one-handed, binaural spreading mechanism that allows a user to force the first binaural away from the second binaural with the use of a single hand, wherein the binaural spreading mechanism comprises:
a first contact member that is selectively attached to the first binaural, wherein the first contact member is sized and shaped such that when it is forced to move in a binaural spreading direction, the first and second binaurals are forced to move away from each other.

18. The stethoscope of claim 17, wherein the binaural spreading direction is selected from:
a) towards a second contact member that is attached to the second binaural,
b) away from the second contact member,
c) towards a binaural fulcrum that connects the first binaural to the second binaural,
d) away from the fulcrum, and
e. towards the second binaural.

19. The stethoscope of claim 17, wherein the binaural spreading direction is selected from:
a) towards a second contact member that is attached to the second binaural, and
b) away from the second contact member.

20. The stethoscope of claim 19, wherein the first and second contact members are sized and shaped such that when the first and second contact members are respectively connected to the first and second binaurals so that the first and second contact members are disposed between the first and second binaurals, the first and second binaurals are forced to move away from each other when the first and second contact members are forced away from each other.
